(19) United States
(12) Patent Application Publication
(10) Pub. No.: US 2022/0259392 A1
(43) Pub. Date: Aug. 18, 2022

(54) HOMOGENEOUS ANION EXCHANGE MEMBRANE AND BIOSENSING MEMBRANE PREPARED FROM THE SAME

(71) Applicant: YUAN ZE UNIVERSITY, Taoyuan City (TW)

(72) Inventors: Yi-Ming Sun, Taoyuan City (TW); Li-Fen Huang, Taoyuan City (TW); Jie-Ning Chuang, Taoyuan City (TW); Wen-Shan Huang, Taoyuan City (TW)

(21) Appl. No.: 17/525,369

(22) Filed: Nov. 12, 2021

(30) Foreign Application Priority Data
Feb. 8, 2021 (TW) 110104812

(51) Int. Cl. C08J 5/22; C08F 2/50; C08F 20/28; C08F 20/32; C12Q 1/6806

(52) U.S. Cl. CPC C08J 5/2243; C08F 2/50; C08F 20/28; C08K 5/07; C12Q 1/6806; C08J 2333/14; C08F 20/32

(57) ABSTRACT

Disclosed herein is a homogeneous anion exchange membrane produced by subjecting a hydrophilic monomer having an ethylenically unsaturated group to free radical polymerization with a quaternary ammonium salt having an ethylenically unsaturated group, a crosslinking agent, and a photoinitiator. A molar ratio of the hydrophilic monomer to the quaternary ammonium salt is in a range from 1:0.1 to 1:0.7. A biosensing membrane prepared from the homogeneous anion exchange membrane is also disclosed.

### CROSS-REFERENCE TO RELATED APPLICATION

This application claims priority of Taiwanese Invention Patent Application No. 110104812, filed on Feb. 8, 2021.

### FIELD

The disclosure relates to a homogeneous anion exchange membrane and a biosensing membrane prepared from the same.

### BACKGROUND

Many conventional methods, such as polymerase chain reaction (PCR), reverse transcription-polymerase chain reaction (RT-PCR), real-time quantitative PCR (qPCR), etc., are being accepted as standard techniques for the detection of a target nucleic acid molecule in a sample. However, in order to obtain correct analysis results, precise operation techniques are required to prevent DNA contamination, and a temperature controller needs to be used to accurately control the temperature of the entire operation process.

Anion exchange membranes (AEMs) are semipermeable membranes generally made from ionomers and designed to allow the transportation of anions from the cathode to the anode in an electrochemical reaction. AEMs are widely used in batteries, sensors, and actuators. Recently, several biosensing technologies using AEMs have been developed to detect nucleic acid molecules. For instance, Z. Slouka et al. disclose an integrated, sample-to-answer diagnostic platform for rapid detection of microRNA biomarkers from cancer cell lines. The integrated diagnostic platform consisted of three units including a pre-treatment unit for separation of nucleic acids from lysates, a pre-concentration unit for concentration of isolated nucleic acids, and a sensing unit localized at a designated position on the chip for specific detection of a target nucleic acid. A probe-functionalized heterogeneous anion exchange membrane sensor was used in the integrated diagnostic platform for rapid and sensitive detection of target molecules (Z. Slouka et al. (2015), Talanta, 145:35-42).

A heterogeneous anion exchange membrane as described above is prepared by thermocompression molding of two stacked polyester fiber support layers and polyethylene particles containing quaternary ammonium compounds and packed between the polyester fiber support layers. The polyester fiber support layers do not have ion exchange function, and the polyethylene particles are unevenly distributed between the polyester fiber support layers. Therefore, heterogeneous anion exchange membranes prepared by the above method are different in ion flux, and different regions in the same heterogeneous anion exchange membrane may also be different in ion flux, thereby leading to poor reproducibility of the detection results of the membrane sensor.

### SUMMARY

Accordingly, a first object of the present disclosure is to provide a homogeneous anion exchange membrane that can alleviate at least one of the drawbacks of the prior art.

The homogeneous anion exchange membrane is produced by:

subjecting a hydrophilic monomer having an ethylenically unsaturated group to free radical polymerization with a quaternary ammonium salt having an ethylenically unsaturated group, a crosslinking agent, and a photoinitiator,

wherein a molar ratio of the hydrophilic monomer to the quaternary ammonium salt is in a range from 1:0.1 to 1:0.7.

A second object of the present disclosure is to provide a biosensing membrane which can alleviate at least one of the drawbacks of the prior art, and which is produced by:
a) providing a homogeneous anion exchange membrane obtained by:
   subjecting a hydrophilic monomer having an ethylenically unsaturated group to free radical polymerization with a quaternary ammonium salt having an ethylenically unsaturated group, a crosslinking agent, and a photoinitiator,
   wherein a molar ratio of the hydrophilic monomer to the quaternary ammonium salt is in a range from 1:0.15 to 1:0.25;
b) subjecting the homogeneous anion exchange membrane to a light-induced grafting reaction with a compound having four carboxylic acid groups to obtain a modified homogeneous anion exchange membrane having four carboxylic acid groups;
c) reacting the modified homogeneous anion exchange membrane with a carbodiimide compound to obtain a carbodiimide activated homogeneous anion exchange membrane having four carboxylic acid groups; and
d) reacting the carbodiimide activated homogeneous anion exchange membrane with a nucleic acid probe having amine groups, so that the nucleic acid probe is coupled to the carbodiimide activated homogeneous anion exchange membrane via an amide bond to result in production of the biosensing membrane.

### BRIEF DESCRIPTION OF THE DRAWINGS

The above and other objects, features and advantages of the present disclosure will become apparent with reference to the following detailed description and the exemplary embodiments taken in conjunction with the accompanying drawings, in which:

FIG. 1 shows the Fourier transform infrared spectroscopy (FTIR) spectra of the homogeneous anion exchange membrane of Example 1, infra, diallyldimethylammonium chloride (DDA), and 2-hydroxyethyl methacrylate (HEMA); and

FIG. 2 shows the current-voltage curves of a biosensing membrane before and after DNA hybridization, in which: I₁ represents an intersection point (current) of S_{U} and S_{L} lines; I₂ represents an intersection point (current) of S_{L} and S_{O} lines; V_{I1} represents a voltage of I₁; V_{I2} represents a voltage of I₂; C_{L} represents an average of I₁ and I₂; and ΔW_{L} represents a width of limiting current (V_{I2}−V_{I1}).

### DETAILED DESCRIPTION

It is to be understood that, if any prior art publication is referred to herein, such reference does not constitute an admission that the publication forms a part of the common general knowledge in the art, in Taiwan or any other country.

For the purpose of this specification, it will be clearly understood that the word “comprising” means “including but not limited to”, and that the word “comprises” has a corresponding meaning.

Unless defined otherwise, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

The present disclosure provides a homogeneous anion exchange membrane produced by:

subjecting a hydrophilic monomer having an ethylenically unsaturated group to free radical polymerization with a quaternary ammonium salt having an ethylenically unsaturated group, a crosslinking agent, and a photoinitiator.

According to the present disclosure, the hydrophilic monomer is electrically neutral or positively charged. The hydrophilic monomer may be selected from the group consisting of 2-hydroxyethyl methacrylate (HEMA), 2-hydroxyethyl acrylate (HEA), hydroxypropyl methacrylate, hydroxypropyl acrylate, N-(2-hydroxypropyl) methacrylamide (HPMA), glycerol monomethacrylate, 2-hydroxy-3-phenoxypropyl methacrylate, 4-hydroxybutyl acrylate, N-(2-hydroxyethyl)acrylamide (HEAA), and combinations thereof.

According to the present disclosure, the quaternary ammonium salt may be selected from the group consisting of diallyldimethylammonium chloride, acryloyloxyethyltrimethyl ammonium chloride, and combinations thereof.

According to the present disclosure, the amount of the quaternary ammonium salt ranges from 0.1 mole to 0.7 mole, based on the amount of the hydrophilic monomer which is 1 mole. As the quaternary ammonium salt is present in an amount not lower than 0.1 mole, the homogeneous anion exchange membrane can generate a current-voltage curve. As the quaternary ammonium salt is present in an amount not greater than 0.7 mole, the formation of the homogeneous anion exchange membrane can be facilitated.

In certain embodiments, the amount of the quaternary ammonium salt ranges from 0.15 mole to 0.25 mole, based on the amount of the hydrophilic monomer which is 1 mole.

According to the present disclosure, the crosslinking agent may be selected from the group consisting of ethylene glycol dimethacrylate, allyl methacrylate, N,N-diallylacrylamide, 1,4-phenylene diacrylate, 1,5-pentanediol dimethacrylate, 1,4-butanediol diacrylate, diurethane dimethacrylate (DUDMA), 1,10-decanediol dimethacrylate, 1,3-butanediol dimethacrylate, 1,4-butanediol dimethacrylate, 1,9-nonanediol dimethacrylate, 1,6-hexanediol dimethacrylate, triethylene glycol dimethacrylate, tricyclodecane dimethanol diacrylate, tetraethylene glycol diacrylate, bis(2-methacryloxyethyl) phosphate, diethylene glycol diacrylate, diethylene glycol dimethacrylate, triethylene glycol diacrylate, triethylene glycol dimethacrylate, tetraethylene glycol dimethacrylate, polyethylene glycol dimethacrylate (PEGDMA), polyethylene glycol diacrylate (PEGDA), poly(ethylene glycol)diglycidyl ether (PEGDGE), and combinations thereof.

It should be appreciated that the amount of the crosslinking agent may vary depending on the types and amounts of the hydrophilic monomer and the quaternary ammonium salt. The choice of these conditions can be routinely determined by those skilled in the art on their own.

In certain embodiments, the amount of the crosslinking agent ranges from 0.01 mole to 0.05 mole, based on the amount of the hydrophilic monomer which is 1 mole.

The homogeneous anion exchange membrane according to the present disclosure has a uniform distribution of ion flux and can generate a current-voltage curve, and is capable of improving the efficiency of nucleic acid probe immobilization.

Therefore, the present disclosure further provides a biosensing membrane produced by:
a) providing a homogeneous anion exchange membrane obtained by:
   subjecting a hydrophilic monomer having an ethylenically unsaturated group to free radical polymerization with a quaternary ammonium salt having an ethylenically unsaturated group, a crosslinking agent, and a photoinitiator;
b) subjecting the homogeneous anion exchange membrane to a light-induced grafting reaction with a compound having four carboxylic acid groups to obtain a modified homogeneous anion exchange membrane having four carboxylic acid groups;
c) reacting the modified homogeneous anion exchange membrane with a carbodiimide compound to obtain a carbodiimide activated homogeneous anion exchange membrane having four carboxylic acid groups; and
d) reacting the carbodiimide activated homogeneous anion exchange membrane with a nucleic acid probe having amine groups, so that the nucleic acid probe is coupled to the carbodiimide activated homogeneous anion exchange membrane via an amide bond to result in production of the biosensing membrane.

According to the present disclosure, in preparing the biosensing membrane, the amount of the quaternary ammonium salt ranges from 0.15 mole to 0.25 mole, based on the amount of the hydrophilic monomer which is 1 mole. As the quaternary ammonium salt is present in an amount not lower than 0.15 mole, the biosensing membrane thus obtained is easy to hybridize with nucleic acids in a test sample and can generate a current-voltage curve. As the quaternary ammonium salt is present in an amount not greater than 0.25 mole, the biosensing membrane thus obtained has good dimensional stability, such that such membrane is not easy to swell and deform after adsorbing a liquid sample. Therefore, the biosensing membrane can be firmly disposed on a support of an electrochemical analysis device for use as a sensor head.

According to the present disclosure, the compound having four carboxylic acid groups may be benzophenone-3,3′,4,4′-tetracarboxylic acid.

According to the present disclosure, the carbodiimide compound may be 1-ethyl-3-(3-dimethylaminopropyl) carbodiimide (EDC).

It should be appreciated that the amounts of the compound having four carboxylic acid groups and the carbodiimide compound may vary depending on the volume and weight of the biosensing membrane to be produced. The choice of these conditions can be routinely determined by those skilled in the art on their own.

As used herein, the term “nucleic acid probe” refers to an oligonucleotide, such as a single-stranded molecule or segment of DNA or RNA, capable of binding to a target nucleic acid of complementary sequence through one or more types of chemical bonds, usually through complementary base pairing and hydrogen bonding.

The disclosure will be further described by way of the following examples. However, it should be understood that the following examples are solely intended for the purpose of illustration and should not be construed as limiting the disclosure in practice.

### EXAMPLES

### Examples 1 to 4

### A. Preparation of Homogeneous Anion Exchange Membrane

0.12 mmol of diallyldimethylammonium chloride (DDA) (Tokyo Chemical Industry Co., Ltd.), 0.85 mmol of 2-hydroxyethyl methacrylate (HEMA) (Acros Organics Inc.), 0.01 mmol of ethylene glycol dimethacrylate (EGDMA) (Alfa Aesar Inc.), and a suitable amount of Irgacure 1173 (BASF SE) (serving as a photoinitiator) were mixed homogeneously, so as to form a mixture. The mixture was subjected to a free radical polymerization through ultraviolet (UV) irradiation at a wavelength of 356 nm and an intensity of 120 J/cm² for 25 minutes, so as to obtain a homogeneous anion exchange membrane of Example 1.

Homogeneous anion exchange membranes of Examples 2 to 4 were prepared using the recipe shown in Table 1 and according to the procedures described above.

The homogeneous anion exchange membrane of the respective one of Examples 1 to 4 was immersed in 0.1× phosphate-buffered saline (PBS) (pH 7.2) (Uniregion Bio Tech Inc.) for at least 24 hours to reach the equilibrium degree of swelling for subsequent use.

TABLE 1

| | Example | | | |
|---|---|---|---|---|
| | 1 | 2 | 3 | 4 |
| DDA(mmol) | 0.12 | 0.16 | 0.20 | 0.2 |
| HEMA(mmol) | 0.85 | 0.85 | 0.85 | 0.85 |
| EGDMA(mmol) | 0.01 | 0.01 | 0.01 | 0.01 |
| DDA/HEMA | 0.141 | 0.188 | 0.235 | 0.271 |

### B. Preparation of Biosensing Membrane

The homogeneous anion exchange membrane of the respective one of Examples 1 to 4 obtained in Section A was taken out from 0.1× PBS (pH=7.2) and was cut to have a size of 0.25 mm².

2 mg of benzophenone-3,3′,4,4′-tetracarboxylic acid (Sigma Aldrich) was dissolved in 50 μL of deionized water, and the resultant mixture was adjusted to pH 7 through addition of sodium hydroxide to obtain a carboxylic acid solution. 20 μL of the carboxylic acid solution was added to the surface of the respective homogeneous anion exchange membrane, followed by conducting a light-induced grafting reaction through UV irradiation at a wavelength of 245 nm and an intensity of 120 J/cm² for 10 minutes, so as to obtain a modified homogeneous anion exchange membrane having four carboxylic acid groups. The modified homogeneous anion exchange membrane was immersed in 0.1× PBS (pH 2) for 8 hours, followed by immersing in 0.1× PBS (pH 7) for subsequent use.

11.4 mg of 1-ethyl-3-(3-dimethylaminopropyl)carbodiimide (EDC) (Sigma Aldrich) was dissolved in 150 μL of MES buffer (pH=5.5) to obtain an EDC solution. The modified homogeneous anion exchange membrane was immersed in the EDC solution for 45 minutes, so as to obtain a EDC-activated homogeneous anion exchange membrane having four carboxylic acid groups. Thereafter, the EDC-activated homogeneous anion exchange membrane was immersed in 20 μL of a 10 μM DNA probe solution (containing DNA probes having amine groups at the 5′-terminus) (Integrated DNA Technologies Inc.), followed by standing at 4° C. for 24 hours, so as to obtain a biosensing membrane.

The biosensing membrane of the respective one of Examples 1 to 4 was immersed in 0.1× PBS (pH 7) for subsequent use.

### Comparative Example 1

A commercial heterogeneous anion exchange membrane (Ralex®, MEGA a.s., Czech Republic) was used as a heterogeneous anion exchange membrane of Comparative Example 1. The heterogeneous anion exchange membrane was composed of a polymeric membrane made by hot pressing polvstyrene-divinylbenzene particles having strong alkaline quaternary ammonium groups, polyamide/polyester fabrics for supporting the polymeric membrane, and a polyethylene adhesive layer for bonding the polymeric membrane and the polyamide/polyester fabrics.

The biosensing membrane of Comparative Example 1 was prepared using the heterogeneous anion exchange membrane according to the procedures described in Section B above.

### Characteristic Analysis of Homogeneous Anion Exchange Membrane and Biosensing Membrane:

### A. Chemical Structure Analysis

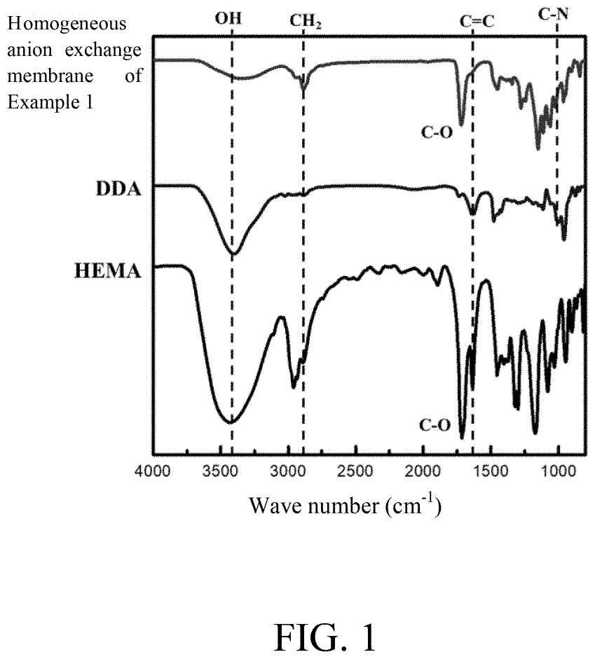

A Fourier-transform infrared spectroscopy (FTIR) instrument (Spectrum 100, Perkin-Elmer) was used to analyze the functional groups of the homogeneous anion exchange membrane of Example 1. The operation conditions for FTIR are as follows: scanning range: 4000 to 650 cm⁻¹; resolution: 4 cm⁻¹; and number of scans: 64 times. The result is shown in FIG. 1.

### B. Elemental Analysis

The homogeneous anion exchange membrane and biosensing membrane of the respective one of Examples 2 to 3 and the heterogeneous anion exchange membrane and biosensing membrane of Comparative Example 1 were subjected to elemental analysis using an X-ray photoelectron spectrometer (VG ESCALAB 250, Thermo Fisher Scientific). The spectra thus obtained were calibrated using the C1s spectrum (284.5 Ev), and were analyzed by XPSPEAK4 software.

In addition, the C—N area increase rate was calculated using the following Equation (I):

C—N area increase rate (%)=[(A−B)/B]×100 (I)

where A=C—N area of respective biosensing membrane
B=C—N area of corresponding anion exchange membrane

The results are shown in Table 2 below.

### C. Measurement of Ion Exchange Capacity (IEC)

0.7 g of the homogeneous anion exchange membrane of the respective one of Examples 1 to 4 and the heterogeneous anion exchange membrane of Comparative Example 1 were dried in an oven for 24 hours, followed by weighing to obtain the dry weight of the respective anion exchange membrane. The respective anion exchange membrane was immersed in 100 mL of a 1 M HCl aqueous solution for 24 hours to adsorb chloride ions, followed by washing with deionized water to remove excess chloride ions until the equivalence point was reached. The respective anion exchange membrane adsorbing chloride ions was immersed in 50 mL of a 1M Na₂SO₄ aqueous solution for 24 hours, so that the chloride ions on the anion exchange membrane were replaced by sulfate ions. Thereafter, the anion exchange membrane was removed, and the resulting solution was collected. 0.1 M K₂CrO₄ (serving as an indicator) was added to the solution, followed by titration with a 0.1 M AgNO₃ aqueous solution.

The IEC was calculated using the following Equation (II):

C=(D×E)/F (II)

where C=IEC (mmol/g)
D=volume of AgNO₃ aqueous solution (mL)
E=concentration of AgNO₃ aqueous solution (mmol/mL)
F=dry weight of respective anion exchange membrane (g)

The result is shown in Table 3 below.

### D. Determination of Swelling Degree and Water Absorption Rate

The homogeneous anion exchange membrane of the respective one of Examples 1 to 4 and the heterogeneous anion exchange membrane of Comparative Example 1 were cut to have a size of 0.5 mm (length)×0.5 mm (width)×0.25 mm (height). The respective anion exchange membrane was dried in an oven for 24 hours, followed by measuring the dry weight and thickness. Thereafter, the respective dried anion exchange membrane was immersed in deionized water for 24 hours, followed by measuring the wet weight and thickness.

The water absorption rate (I) was calculated using the following Equation (III):

G=[(H−I)/I]×100 (III)

where G=water absorption rate (%)
H=wet weight of respective anion exchange membrane (g)
I=dry weight of respective anion exchange membrane (g)

The swelling degree (%) was calculated using the following Equation (IV):

J=[(K−L)/L]×100 (IV)

where J=swelling degree (%)
K=thickness of respective wetted anion exchange membrane (mm)
L=thickness of corresponding dried anion exchange membrane (mm)

The results are shown in Table 3 below.

### E. Measurement of Offset Voltage (ΔV)

The biosensing membrane of the respective one of Examples 1 to 4 and Comparative Example 1 was cut to have a size of 0.25 mm². The respective biosensing membrane was disposed on a holder made of PU resin (TAP Plastics) for use as a sensor head. The sensor head was installed in an electrochemical analysis device. The electrochemical analysis device included a microchannel for flow of a test sample, and a first reservoir, a second reservoir, and a third reservoir disposed in the microchannel and spaced apart from one another. The sensor head was disposed at the bottom of the first reservoir and served to directly contact the test sample flowing into the microchannel.

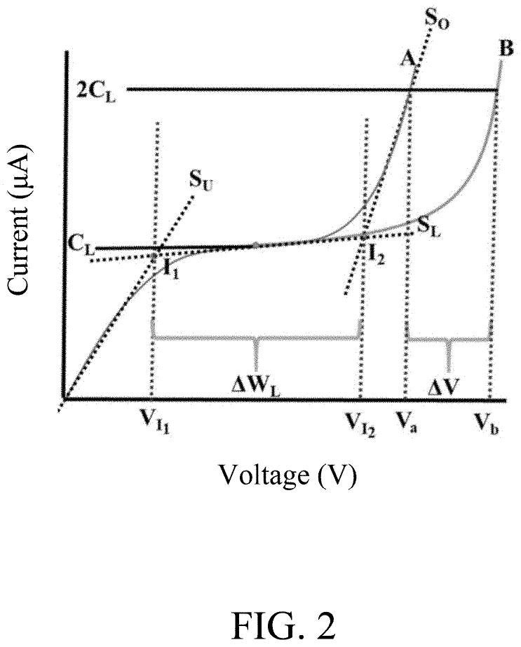

A respective one of the three reservoirs was filled with 0.1× PBS buffer. A first platinum electrode and a first silver/silver chloride (Ag/AgCl) reference electrode were disposed in the first reservoir, a second platinum electrode was disposed in the second reservoir, and a second silver/silver chloride reference electrode was disposed in the third reservoir. 0.1× PBS serving as a test sample was allowed to pass through the microchannel. The first platinum electrode and the second platinum electrode provided a current of 0-100 μA at a rate of 1 μA/s, and the first silver/silver chloride reference electrode and the second silver/silver chloride reference electrode measured the potential, thereby obtaining an initial current-voltage curve of the biosensing membrane. Referring to FIG. 2, the initial current-voltage curve is labelled with A.

Thereafter, a solution containing 200 ng/μL soybean nucleic acid was used as a test sample, and the current-voltage curve generated after DNA hybridization was obtained according to the procedures described above. Referring to FIG. 2, the current-voltage curve generated by DNA hybridization is labelled with B.

The offset voltage (ΔV) was calculated using the following Equation (V):

ΔV=V_{b}−Vₐ (V)

where V_{b}=voltage corresponding to the current-voltage curve B at two-fold limiting current (V)
Vₐ=voltage corresponding to the current-voltage curve A at two-fold limiting current (V)

The limiting current (C_{L}) is defined as the average current applied, namely, C_{L}=(I₁+I₂)/2.

The result is shown in Table 3.

### F. Measurement of Reproducibility

Pieces (n=3) of the homogeneous anion exchange membrane of the respective one of Examples 1 to 3 and pieces (n=3) of the heterogeneous anion exchange membrane of Comparative Example 1 were used as test membrane samples. The respective piece was disposed on a support as described in section E above for use as a sensor head. The sensor head was disposed in an electrochemical analysis device as described in section E above, and the procedures described in the abovementioned section E were performed to obtain a current-voltage curve. The slopes S_{U} (i.e., a slope of an under-limiting region), S_{L} (i.e., a slope of a limiting region), and S_{O} (i.e., a slope of an over-limiting region) of the respective current-voltage curve were calculated, and the experimental data are expressed as mean±SD (standard deviation).

The result is shown in Table 3 below.

### Results:

Referring to FIG. 1, the FTIR spectrum of the homogeneous anion exchange membrane of Example 1 was different from those of HEMA and DDA. In particular, the C═C stretching peak at 1625 cm⁻¹ of the homogeneous anion exchange membrane of Example 1 was significantly reduced, indicating the successful polymerization of HEMA and DDA. In addition, the homogeneous anion exchange membrane of Example 1 had a C—N stretching peak at 1040 cm⁻¹, indicating that the homogeneous anion exchange membrane of Example 1 had quaternary ammonium groups derived from DDA.

As shown in Table 2 below, for each of Examples 2 and 3, the N element content and C—N area of the biosensing membrane were higher than those of the corresponding homogeneous anion exchange membrane used to prepare the biosensing membrane, indicating that the DNA probe was effectively immobilized on the biosensing membrane. In addition, The C—N area increase rates of Examples 2 and 3 were 19.1% and 26.8%, respectively, and the C—N area increase rate of Comparative Example 1 was 5.4%. These results indicate that as compared to the heterogeneous anion exchange membrane of Comparative Example 1, the homogeneous anion exchange membranes of Examples 2 to 3 had better binding efficiency with DNA probes.

As shown in Table 3 below, the swelling degrees of the homogeneous anion exchange membranes of Examples 1 to 3 were lower than that of the heterogeneous anion exchange membrane of Comparative Example 1, indicating that the homogeneous anion exchange membranes of Examples 1 to 3 had better dimensional stability. In addition, when the amount of DDA was increased, the IEC and water absorption rate of the exemplary homogeneous anion exchange membranes of the present invention were increased.

Furthermore, the standard deviations of determined in the homogeneous anion exchange membranes of Examples 1 to 3 were lower than that determined in the heterogeneous anion exchange membrane of Comparative Example 1, and similar results were observed with respect to the standard deviations of S_{L} and S_{O}. These results indicate that as compared to the heterogeneous anion exchange membrane of Comparative Example 1, the homogeneous anion exchange membranes of Examples 1 to 3 had stable ion flux, and hence the current-voltage characteristic curves produced therefrom had better reproducibility.

Moreover, the offset voltages produced by the biosensing membranes of Examples 2 to 3 were higher than that of the biosensing membrane of Comparative Example 1, indicating that the biosensing membranes of Examples 2 to 3 had better detection sensitivity.

TABLE 2

| | | Element | | | C₁ₛ area (%) | | | |
|---|---|---|---|---|---|---|---|---|
| | | component (%) | | | C—C/ | | | |
| | | C | N | O | C═C | C—N | C—O—C | O—C═O |
| Comparative | Heterogeneous | 94.4 | 1.7 | 3.9 | 63.0 | 29.9 | 5.9 | 1.2 |
| Example 1 | anion exchange | | | | | | | |
| | membrane | | | | | | | |
| | Biosensing | 90.0 | 2.9 | 7.1 | 60.2 | 31.5 | 5.8 | 2.5 |
| | membrane | | | | | | | |
| Example 2 | Homogeneous | 75.0 | 1.4 | 23.6 | 51.8 | 19.9 | 18.0 | 10.3 |
| | anion exchange | | | | | | | |
| | membrane | | | | | | | |
| | Biosensing | 72.7 | 4.9 | 22.4 | 44.0 | 23.7 | 21.8 | 10.5 |
| | membrane | | | | | | | |
| Example 3 | Homogeneous | 74.5 | 1.8 | 23.7 | 47.8 | 22.8 | 19.4 | 10.0 |
| | anion exchange | | | | | | | |
| | membrane | | | | | | | |
| | Biosensing | 67.6 | 6.9 | 25.5 | 42.2 | 28.9 | 19.1 | 9.8 |
| | membrane | | | | | | | |

TABLE 3

| | | | | | | Comparative |
|---|---|---|---|---|---|---|
| | | Example | | | | Example |
| | | 1 | 2 | 3 | 4 | 1 |
| Anion | IEC | 0.70 | 0.83 | 1.10 | 1.65 | 1.72 |
| exchange | (mmol/g) | | | | | |
| membrane | Water | 58 | 63 | 71 | 98 | 65 |
| | absorption | | | | | |
| | rate (%) | | | | | |
| | Swelling | 22 | 33 | 50 | 76 | 60 |
| | degree (%) | | | | | |
| | S_{U} | 25.16667 ± 3.36386 | 34.96667 ± 0.94281 | 51.7 ± 0.94163 | Not | 76.66667 ± 13.15303 |
| | | | | | determined | |
| | S_{L} | 3.86667 ± 0.4714 | 4.83333 ± 0.67987 | 6.46667 ± 0.12472 | Not | 6.5 ± 1.2083 |
| | | | | | determined | |
| | S_{O} | 20.66667 ± 1.34743 | 19.26667 ± 0.28674 | 30.46667 ± 0.80554 | Not | 19.66667 ± 4.40631 |
| | | | | | determined | |
| Biosensing | ΔV (V) | Not | 0.29 | 0.16 | Not | 0.13 |
| membrane | | determined | | | determined | |

In the description above, for the purposes of explanation, numerous specific details have been set forth in order to provide a thorough understanding of the embodiments. It will be apparent, however, to one skilled in the art, that one or more other embodiments maybe practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A homogeneous anion exchange membrane produced by:
subjecting a hydrophilic monomer having an ethylenically unsaturated group to free radical polymerization with a quaternary ammonium salt having an ethylenically unsaturated group, a crosslinking agent, and a photoinitiator,
wherein a molar ratio of the hydrophilic monomer to the quaternary ammonium salt is in a range from 1:0.1 to 1:0.7.

2. The homogeneous anion exchange membrane according to claim 1, wherein the molar ratio of the hydrophilic monomer to the quaternary ammonium salt is in a range from 1:0.15 to 1:0.25.

3. The homogeneous anion exchange membrane according to claim 1, wherein the quaternary ammonium salt is selected from the group consisting of diallyldimethylammonium chloride, acryloyloxyethyltrimethyl ammonium chloride, and combinations thereof.

4. The homogeneous anion exchange membrane according to claim 1, wherein the hydrophilic monomer is selected from the group consisting of 2-hydroxyethyl methacrylate, 2-hydroxyethyl acrylate, hydroxypropyl methacrylate, hydroxypropyl acrylate, N-(2-hydroxypropyl) methacrylamide, glycerol monomethacrylate, 2-hydroxy-3-phenoxypropyl methacrylate, 4-hydroxybutyl acrylate, N-(2-hydroxyethyl)acrylamide, and combinations thereof.

5. The homogeneous anion exchange membrane according to claim 1, wherein the crosslinking agent is selected from the group consisting of ethylene glycol dimethacrylate, allyl methacrylate, N,N-diallylacrylamide, 1,4-phenylene diacrylate, 1,5-pentanediol dimethacrylate, 1,4-butanediol diacrylate, diurethane dimethacrylate, 1,10-decanediol dimethacrylate, 1,3-butanediol dimethacrylate, 1,4-butanediol dimethacrylate, 1,9-nonanediol dimethacrylate, 1,6-hexanediol dimethacrylate, triethylene glycol dimethacrylate, tricyclodecane dimethanol diacrylate, tetraethylene glycol diacrylate, bis(2-methacryloxyethyl) phosphate, diethylene glycol diacrylate, diethylene glycol dimethacrylate, triethylene glycol diacrylate, triethylene glycol dimethacrylate, tetraethylene glycol dimethacrylate, polyethylene glycol dimethacrylate, polyethylene glycol diacrylate, poly(ethylene glycol)diglycidyl ether, and combinations thereof.

6. A biosensing membrane produced by:
a) providing a homogeneous anion exchange membrane obtained by:
subjecting a hydrophilic monomer having an ethylenically unsaturated group to free radical polymerization with a quaternary ammonium salt having an ethylenically unsaturated group, a crosslinking agent, and a photoinitiator,
wherein a molar ratio of the hydrophilic monomer to the quaternary ammonium salt is in a range from 1:0.15 to 1:0.25;
b) subjecting the homogeneous anion exchange membrane to a light-induced grafting reaction with a compound having four carboxylic acid groups to obtain a modified homogeneous anion exchange membrane having four carboxylic acid groups;
c) reacting the modified homogeneous anion exchange membrane with a carbodiimide compound to obtain a carbodiimide activated homogeneous anion exchange membrane having four carboxylic acid groups; and
d) reacting the carbodiimide activated homogeneous anion exchange membrane with a nucleic acid probe having amine groups, so that the nucleic acid probe is coupled to the carbodiimide activated homogeneous anion exchange membrane via an amide bond to result in production of the biosensing membrane.

7. The biosensing membrane according to claim 6, wherein the compound having four carboxylic acid groups is benzophenone-3,3′,4,4′-tetracarboxylic acid.

8. The biosensing membrane according to claim 6, wherein the carbodiimide compound is 1-ethyl-3-(3-dimethylaminopropyl)carbodiimide.
